(19) United States
(12) Patent Application Publication
(10) Pub. No.: US 2007/0053888 A1
(43) Pub. Date: Mar. 8, 2007

(54) Use of umbilical cord blood to treat individuals having a disease, disorder or condition

(76) Inventor: Robert J. Hariri, Florham Park, NJ (US)

Correspondence Address: JONES DAY, 222 EAST 41ST ST, NEW YORK, NY, 10017, US

(21) Appl. No.: 11/593,348

(22) Filed: Nov. 6, 2006

Related U.S. Application Data
(63) Continuation of application No. 10/779,369, filed on Feb. 13, 2004.
(60) Provisional application No. 60/447,252, filed on Feb. 13, 2003.

(51) Int. Cl. A61K 35/14; C12N 5/08

(52) U.S. Cl. 424/93.7; 435/372

(57) ABSTRACT

The present invention provides methods of using cord blood and cord blood-derived stem cells in high doses to treat various conditions, diseases and disorders. The high-dose cord blood and cord blood-derived stem cells have a multitude of uses and applications, including but not limited to, therapeutic uses for transplantation and treatment and prevention of disease, and diagnostic and research uses. In particular, the cord blood or cord blood-derived stem cells are delivered in high doses, e.g., at least 3 billion nucleated cells per treatment, where treatment may comprise a single or multiple infusions. The invention also provides for the use of cord blood or cord blood-derived stem cells from multiple donors without the need for HLA typing.

This application claims benefit of U.S. Provisional Application No. 60/447,252, filed Feb. 13, 2003, which is incorporated herein by reference in its entirety.

### 1. INTRODUCTION

The present invention relates to the use of cord blood compositions in large doses and without pre-transfusion HLA typing. Cord blood has a multitude of uses and applications, including but not limited to, therapeutic uses for transplantation, diagnostic and research uses. In particular, cord blood is useful in the treatment of diseases or disorders, including vascular disease, neurological diseases or disorders, autoimmune diseases or disorders, and diseases or disorders involving inflammation.

### 2. BACKGROUND OF THE INVENTION

There is considerable interest in the identification, isolation and generation of human stem cells. Human stem cells are totipotential or pluripotential precursor cells capable of generating a variety of mature human cell lineages. This ability serves as the basis for the cellular differentiation and specialization necessary for organ and tissue development.

Recent success at transplanting such stem cells have provided new clinical tools to reconstitute and/or supplement bone marrow after myeloablation due to disease, exposure to toxic chemical and/or radiation. Further evidence exists that demonstrates that stem cells can be employed to repopulate many, if not all, tissues and restore physiologic and anatomic functionality. The application of stem cells in tissue engineering, gene therapy delivery and cell therapeutics is also advancing rapidly.

Many different types of mammalian stem cells have been characterized. For example, embryonic stem cells, embryonic germ cells, adult stem cells or other committed stem cells or progenitor cells are known. Certain stem cells have not only been isolated and characterized but have also been cultured under conditions to allow differentiation to a limited extent. A basic problem remains, however, in that obtaining sufficient quantities and populations of human stem cells which are capable of differentiating into all cell types is near impossible. The provision of matched stem cell units of sufficient quantity and quality remains a challenge despite the fact that these are important for the treatment of a wide variety of disorders, including malignancies, inborn errors of metabolism, hemoglobinopathies, and immunodeficiencies.

Umbilical cord blood (“cord blood”) is a known alternative source of hematopoietic progenitor stem cells. Stem cells from cord blood are routinely cryopreserved for use in hematopoietic reconstitution, a widely used therapeutic procedure used in bone marrow and other related transplantations (see e.g., Boyse et al., U.S. Pat. No. 5,004,681, “Preservation of Fetal and Neonatal Hematopoietic Stem and Progenitor Cells of the Blood”, Boyse et al., U.S. Pat. No. 5,192,553, entitled “Isolation and preservation of fetal and neonatal hematopoietic stem and progenitor cells of the blood and methods of therapeutic use”, issued Mar. 9, 1993). Conventional techniques for the collection of cord blood are based on the use of a needle or cannula, which is used with the aid of gravity to drain cord blood from (i.e., exsanguinate) the placenta (Boyse et al., U.S. Pat. No. 5,192,553, issued Mar. 9, 1993; Boyse et al., U.S. Pat. No. 5,004,681, issued Apr. 2, 1991; Anderson, U.S. Pat. No. 5,372,581, entitled Method and apparatus for placental blood collection, issued Dec. 13, 1994; Hessel et al., U.S. Pat. No. 5,415,665, entitled Umbilical cord clamping, cutting, and blood collecting device and method, issued May 16, 1995). The needle or cannula is usually placed in the umbilical vein and the placenta is gently massaged to aid in draining cord blood from the placenta. Thereafter, however, the drained placenta has been regarded as having no further use and has typically been discarded. A major limitation of stem cell procurement from cord blood, moreover, has been the frequently inadequate volume of cord blood obtained, resulting in insufficient cell numbers to effectively reconstitute bone marrow after transplantation.

Naughton et al. (U.S. Pat. No. 5,962,325 entitled “Three-dimensional stromal tissue cultures” issued Oct. 5, 1999) discloses that fetal cells, including fibroblast-like cells and chondrocyte-progenitors, may be obtained from umbilical cord or placenta tissue or umbilical cord blood.

Currently available methods for the ex vivo expansion of cell populations are also labor-intensive. For example, Emerson et al. (U.S. Pat. No. 6,326,198 entitled “Methods and compositions for the ex vivo replication of stem cells, for the optimization of hematopoietic progenitor cell cultures, and for increasing the metabolism, GM-CSF secretion and/or IL-6 secretion of human stromal cells”, issued Dec. 4, 2001); discloses methods, and culture media conditions for ex vivo culturing of human stem cell division and/or the optimization of human hematopoietic progenitor stem cells. According to the disclosed methods, human stem cells or progenitor cells derived from bone marrow are cultured in a liquid culture medium that is replaced, preferably perfused, either continuously or periodically, at a rate of 1 ml of medium per ml of culture per about 24 to about 48 hour period. Metabolic products are removed and depleted nutrients replenished while maintaining the culture under physiologically acceptable conditions.

Kraus et al (U.S. Pat. No. 6,338,942, entitled “Selective expansion of target cell populations”, issued Jan. 15, 2002) discloses that a predetermined target population of cells may be selectively expanded by introducing a starting sample of cells from cord blood or peripheral blood into a growth medium, causing cells of the target cell population to divide, and contacting the cells in the growth medium with a selection element comprising binding molecules with specific affinity (such as a monoclonal antibody for CD34) for a predetermined population of cells (such as CD34 cells), so as to select cells of the predetermined target population from other cells in the growth medium.

Rodgers et al. (U.S. Pat. No. 6,335,195 entitled “Method for promoting hematopoietic and mesenchymal cell proliferation and differentiation,” issued Jan. 1, 2002) discloses methods for ex vivo culture of hematopoietic and mesenchymal stem cells and the induction of lineage-specific cell proliferation and differentiation by growth in the presence of angiotensinogen, angiotensin I (AI), AI analogues, AI fragments and analogues thereof, angiotensin II (AII), AII analogues, AII fragments or analogues thereof or AII AT₂ type 2 receptor agonists, either alone or in combination with other growth factors and cytokines. The stem cells are derived from bone marrow, peripheral blood or umbilical cord blood. The drawback of such methods, however, is that such ex vivo methods for inducing proliferation and differentiation of stem cells are time-consuming, as discussed above, and also result in low yields of stem cells.

Naughton et al. (U.S. Pat. No. 6,022,743 entitled “Three-dimensional culture of pancreatic parenchymal cells cultured living stromal tissue prepared in vitro,” issued Feb. 8, 2000) discloses a tissue culture system in which stem cells or progenitor cells (e.g., stromal cells such as those derived from umbilical cord cells, placental cells, mesenchymal stem cells or fetal cells) are propagated on three-dimensional support rather than as a two-dimensional monolayer in, e.g., a culture vessel such as a flask or dish.

Because of restrictions on the collection and use of stem cells, and the inadequate numbers of cells typically collected from cord blood, stem cells are in critically short supply. Stem cells have the potential to be used in the treatment of a wide variety of disorders, including malignancies, inborn errors of metabolism, hemoglobinopathies, and immunodeficiencies. There is a critical need for a readily accessible source of large numbers of human stem cells for a variety of therapeutic and other medically related purposes. The present invention addresses that need and others.

Additionally, the compositions of the invention are expected to be useful in the treatment of neurological conditions such as amylotrophic lateral sclerosis (ALS). Several recent studies using irradiated mouse models of familial ALS, a less-common form of ALS, have suggested that cord blood may be useful in the treatment of this disease. See Ende et al., Life Sci. 67:53059 (2000).

### 3. SUMMARY OF THE INVENTION

The present invention provides a method of treating an individual comprising administering to said individual umbilical cord blood or cellular fraction therefrom, alone or in combination with cells derived from other sources including the placenta. The umbilical cord blood is provided to an individual in high doses, i.e., 5-25×10⁹ total nucleated cells per individual per administration. The method of the invention also specifies that the cord blood may be pooled from a plurality of different sources, without specific need to match HLA type between recipient and donor(s).

The present invention relates to the use of cord blood compositions or stem or progenitor cells therefrom to treat diseases, disorders or conditions. Such diseases, disorders or conditions may be autoimmune in nature or include inflammation as a symptom, and may affect any organ or tissue of the body, particularly the nervous system or vascular system.

In one embodiment, the invention provides a method of treating a patient in need thereof comprising administration of a plurality of umbilical cord blood cells. In a specific embodiment, said patient has or suffers from a neurological disease, disorder or condition. In a more specific embodiment, said disease, disorder or condition is one affecting the central nervous system. In an even more specific embodiment, said disease, disorder or condition is amylotrophic lateral sclerosis. In another even more specific embodiment, said disease, disorder or condition is multiple sclerosis. In another more specific embodiment, said disease, disorder or condition is one affecting the peripheral nervous system. In another more specific embodiment, said disease, disorder or condition is one affecting the vascular system. In another more specific embodiment, said disease, disorder or condition is one involving or caused by inflammation. In another more specific embodiment, said disease, disorder or condition is an autoimmune disease, disorder or condition.

In another embodiment, the invention provides a method of treating myelodysplasia which comprises administering umbilical cord blood cells (or stem cells isolated therefrom) to a patient in need thereof.

### 3.1. Definitions

As used herein, the term “allogeneic cell” refers to a “foreign” cell, i.e., a heterologous cell (i.e., a “non-self” cell derived from a source other than the placental donor) or autologous cell (i.e., a “self” cell derived from the placental donor) that is derived from an organ or tissue other than the placenta.

As used herein, the term “progenitor cell” refers to a cell that is committed to differentiate into a specific type of cell or to form a specific type of tissue.

As used herein, the term “stem cell” refers to a master cell that can differentiate indefinitely to form the specialized cells of tissues and organs. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells.

As used herein, the term “cord blood derived stem cell” includes cord blood-derived progenitor cells, unless otherwise specifically noted.

### 4. DETAILED DESCRIPTION OF THE INVENTION

The present invention is based in part on the unexpected discovery on the part of the inventor that cord blood may be administered to individuals in high doses and without the need for HLA typing. This is surprising, because tissue transplants typically involve the careful matching of donor and recipient tissue types to permit successful, durable engraftment of allogeneic cells in a recipient and to reduce the incidence of graft-versus-host disease (GvHD). This greatly facilitates the collection of cord blood from multiple donors for administration to a single individual. The high-dose administration allows for the provision of enough cord blood-derived stem cells to provide a high likelihood of long-term engraftment of the administered cells. In accordance with the present invention, the high-dose cord blood has a multitude of uses and applications, including but not limited to, therapeutic uses for transplantation and treatment and prevention of disease, and diagnostic and research uses.

The present invention also provides methods of treating the cord blood with a growth factor, e.g., a cytokine and/or an interleukin, to induce cell differentiation.

The present invention provides pharmaceutical compositions that comprise cord blood alone or in combination with cells from the placenta. According to the invention, populations of stem cells from umbilical cord blood have a multitude of uses, including therapeutic and diagnostic uses. The stem cells can be used for transplantation or to treat or prevent disease. In one embodiment of the invention, the cord blood or cord blood-derived stem cells are used to renovate and repopulate tissues and organs, thereby replacing or repairing diseased tissues, organs or portions thereof. In another embodiment, the cord blood or cord blood-derived stem cells can be used as a diagnostic to screen for genetic disorders or a predisposition for a particular disease or disorder.

The present invention also provides methods of treating a patient in need thereof by administration of cord blood or cord blood-derived stem cells.

### 4.1. Collection of Umbilical Cord Blood

Umbilical cord blood may be collected in any medically or pharmaceutically-acceptable manner. Various methods for the collection of cord blood have been described. See, e.g., Coe, U.S. Pat. No. 6,102,871; Haswell, U.S. Pat. No. 6,179,819 B1. Cord Blood may be collected into, for example, blood bags, transfer bags, or sterile plastic tubes. Cord blood or stem cells derived therefrom may be stored as collected from a single individual (i.e., as a single unit) for administration, or may be pooled with other units for later administration.

### 4.2. Cord Blood-Derived Stem Cells

Cord blood-derived stem cells obtained in accordance with the methods of the invention may include pluripotent cells, i.e., cells that have complete differentiation versatility, that are self-renewing, and can remain dormant or quiescent within tissue. Cord blood contains predominantly CD34+ and CD38+ hematopoietic progenitor cells, as well as smaller populations of more undifferentiated or primitive stem cells.

The cord blood-derived stem cells obtained by the methods of the invention may be induced to differentiate along specific cell lineages, including hematopoietic, vasogenic, neurogenic, and hepatogenic. In certain embodiments, cord blood-derived stem cells are induced to differentiate for use in transplantation and ex vivo treatment protocols. In certain embodiments, cord blood-derived stem cells obtained by the methods of the invention are induced to differentiate into a particular cell type and genetically engineered to provide a therapeutic gene product.

Cord blood-derived stem cells may also be further cultured after collection using methods well known in the art, for example, by culturing on feeder cells, such as irradiated fibroblasts, or in conditioned media obtained from cultures of such feeder cells, in order to obtain continued long-term cultures. The stem cells may also be expanded, either before collection or in vitro after collection. In certain embodiments, the stem cells to be expanded are exposed to, or cultured in the presence of, an agent that suppresses cellular differentiation. Such agents are well-known in the art and include, but are not limited to, human Delta-1 and human Serrate-1 polypeptides (see, Sakano et al., U.S. Pat. No. 6,337,387 entitled “Differentiation-suppressive polypeptide”, issued Jan. 8, 2002), leukemia inhibitory factor (LIF) and stem cell factor. Methods for the expansion of cell populations are also known in the art (see e.g., Emerson et al., U.S. Pat. No. 6,326,198 entitled “Methods and compositions for the ex vivo replication of stem cells, for the optimization of hematopoietic progenitor cell cultures, and for increasing the metabolism, GM-CSF secretion and/or IL-6 secretion of human stromal cells”, issued Dec. 4, 2001; Kraus et al., U.S. Pat. No. 6,338,942, entitled “Selective expansion of target cell populations”, issued Jan. 15, 2002).

The cord blood-derived stem cells may be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT cell proliferation assay (to assess proliferation). Longevity may be determined by methods well known in the art, such as by determining the maximum number of population doubling in an extended culture.

Agents that can induce stem or progenitor cell differentiation are well known in the art and include, but are not limited to, Ca²⁺, EGF, α-FGF, β-FGF, PDGF, keratinocyte growth factor (KGF), TGF-β, cytokines (e.g., IL-1α, IL-1β, IFN-γ, TFN), retinoic acid, transferrin, hormones (e.g., androgen, estrogen, insulin, prolactin, triiodothyronine, hydrocortisone, dexamethasone), sodium butyrate, TPA, DMSO, NMF, DMF, matrix elements (e.g., collagen, laminin, heparan sulfate, Matrigel™), or combinations thereof. In certain embodiments, cord blood-derived stem or progenitor cells are induced to differentiate into a particular cell type, by exposure to a growth factor, according to methods well known in the art. In specific embodiments, the growth factor is: GM-CSF, IL-4, Flt3L, CD40L, IFN-alpha, TNF-alpha, IFN-gamma, IL-2, IL-6, retinoic acid, basic fibroblast growth factor, TGF-beta-1, TGF-beta-3, hepatocyte growth factor, epidermal growth factor, cardiotropin-1, angiotensinogen, angiotensin I (AI), angiotensin II (AII), AII AT₂ type 2 receptor agonists, or analogs or fragments thereof.

Agents that suppress cellular differentiation are also well-known in the art and include, but are not limited to, human Delta-1 and human Serrate-1 polypeptides (see, Sakano et al., U.S. Pat. No. 6,337,387 entitled “Differentiation-suppressive polypeptide”, issued Jan. 8, 2002), leukemia inhibitory factor (LIF), and stem cell factor.

Determination that a stem cell has differentiated into a particular cell type may be accomplished by methods well-known in the art, e.g., measuring changes in morphology and cell surface markers using techniques such as flow cytometry or immunocytochemistry (e.g., staining cells with tissue-specific or cell-marker specific antibodies), by examination of the morphology of cells using light or confocal microscopy, or by measuring changes in gene expression using techniques well known in the art, such as PCR and gene-expression profiling.

In one embodiment, cord blood-derived stem or progenitor cells are induced to differentiate into neurons, according to methods well known in the art, e.g., by exposure to β-mercaptoethanol or to DMSO/butylated hydroxyanisole, according to the methods disclosed in Section 5.1.1.s

In another embodiment, the stem or progenitor cells are induced to differentiate into adipocytes, according to methods well known in the art, e.g., by exposure to dexamethasone, indomethicin, insulin and IBMX, according to the methods disclosed in Section 5.1.2.

In another embodiment, the stem or progenitor cells are induced to differentiate into chondrocytes, according to methods well known in the art, e.g., by exposure to TGF-.beta-3, according to the methods disclosed in Section 5.1.3.

In another embodiment, the stem or progenitor cells are induced to differentiate into osteocytes, according to methods well known in the art, e.g., by exposure to dexamethasone, ascorbic acid-2-phosphate and beta-glycerophosphate, according to the methods disclosed in Section 5.1.4.

In another embodiment, the stem or progenitor cells are induced to differentiate into hepatocytes, according to methods well known in the art, e.g., by exposure to IL-6+/−IL-15, according to the methods disclosed in Section 5.1.5.

In another embodiment, the stem or progenitor cells are induced to differentiate into pancreatic cells, according to methods well known in the art, e.g., by exposure to basic fibroblast growth factor, and transforming growth factor beta-1, according to the methods disclosed in Section 5.1.6.

In another embodiment, the stem or progenitor cells are induced to differentiate into cardiac cells, according to methods well known in the art, e.g., by exposure to retinoic acid, basic fibroblast growth factor, TGF-beta-1 and epidermal growth factor, by exposure to cardiotropin-1 or by exposure to human myocardium extract, according to the methods disclosed in Section 5.1.7.

In another embodiment, the stem cells are stimulated to proliferate, for example, by administration of erythropoietin, cytokines, lymphokines, interferons, colony stimulating factors (CSFs), interferons, chemokines, interleukins, recombinant human hematopoietic growth factors including ligands, stem cell factors, thrombopoeitin (Tpo), interleukins, and granulocyte colony-stimulating factor (G-CSF) or other growth factors.

A vector containing a transgene can be introduced into a stem cell of interest by methods well known in the art, e.g., transfection, transformation, transduction, electroporation, infection, microinjection, cell fusion, DEAE dextran, calcium phosphate precipitation, liposomes, LIPOFECTIN™, lysosome fusion, synthetic cationic lipids, use of a gene gun or a DNA vector transporter, such that the transgene is transmitted to daughter cells. For various techniques for transformation or transfection of mammalian cells, see Keown et al., 1990, Methods Enzymol. 185: 527-37; Sambrook et al., 2001, Molecular Cloning, A Laboratory Manual, Third Edition, Cold Spring Harbor Laboratory Press, N.Y.

Preferably, the transgene is introduced using any technique, so long as it is not destructive to the cell's nuclear membrane or other existing cellular or genetic structures. In certain embodiments, the transgene is inserted into the nucleic genetic material by microinjection. Microinjection of cells and cellular structures is commonly known and practiced in the art.

For stable transfection of cultured mammalian cells, only a small fraction of cells may integrate the foreign DNA into their genome. The efficiency of integration depends upon the vector and transfection technique used. In order to identify and select integrants, a gene that encodes a selectable marker (e.g., for resistance to antibiotics) is generally introduced into the stem cell along with the gene sequence of interest. Preferred selectable markers include those that confer resistance to drugs, such as G418, hygromycin and methotrexate. Cells stably transfected with the introduced nucleic acid can be identified by drug selection (e.g., cells that have incorporated the selectable marker gene will survive, while the other cells die). Such methods are particularly useful in methods involving homologous recombination in mammalian cells prior to introduction or transplantation of the recombinant cells into a subject or patient.

A number of selection systems may be used to select transformed cord blood-derived stem cells. In particular, the vector may contain certain detectable or selectable markers. Other methods of selection include but are not limited to selecting for another marker such as: the herpes simplex virus thymidine kinase (Wigler et al., 1977, Cell 11: 223), hypoxanthine-guanine phosphoribosyltransferase (Szybalska and Szybalski, 1962, Proc. Natl. Acad. Sci. USA 48: 2026), and adenine phosphoribosyltransferase (Lowy et al., 1980, Cell 22: 817) genes can be employed in tk-, hgprt- or aprt- cells, respectively. Also, antimetabolite resistance can be used as the basis of selection for the following genes: dhfr, which confers resistance to methotrexate (Wigler et al., 1980, Proc. Natl. Acad. Sci. USA 77: 3567; O'Hare et al., 1981, Proc. Natl. Acad. Sci. USA 78: 1527); gpt, which confers resistance to mycophenolic acid (Mulligan and Berg, 1981, Proc. Natl. Acad. Sci. USA 78: 2072); neo, which confers resistance to the aminoglycoside G-418 (Colberre-Garapin et al., 1981, J. Mol. Biol. 150: 1); and hygro, which confers resistance to hygromycin (Santerre et al., 1984, Gene 30: 147).

The transgene may integrate into the genome of the cell of interest, preferably by random integration. In other embodiments the transgene may integrate by a directed method, e.g., by directed homologous recombination (i.e., “knock-in” or “knock-out” of a gene of interest in the genome of cell of interest), Chappel, U.S. Pat. No. 5,272,071; and PCT publication No. WO 91/06667, published May 16, 1991; U.S. Pat. No. 5,464,764; Capecchi et al., issued Nov. 7, 1995; U.S. Pat. No. 5,627,059, Capecchi et al. issued, May 6, 1997; U.S. Pat. No. 5,487,992, Capecchi et al., issued Jan. 30, 1996).

Methods for generating cells having targeted gene modifications through homologous recombination are known in the art. The construct will comprise at least a portion of a gene of interest with a desired genetic modification, and will include regions of homology to the target locus, i.e., the endogenous copy of the targeted gene in the host's genome. DNA constructs for random integration, in contrast to those used for homologous recombination, need not include regions of homology to mediate recombination. Markers can be included in the targeting construct or random construct for performing positive and negative selection for insertion of the transgene.

To create a homologous recombinant cell, e.g., a homologous recombinant cord blood-derived stem cell, a homologous recombination vector is prepared in which a gene of interest is flanked at its 5′ and 3′ ends by gene sequences that are endogenous to the genome of the targeted cell, to allow for homologous recombination to occur between the gene of interest carried by the vector and the endogenous gene in the genome of the targeted cell. The additional flanking nucleic acid sequences are of sufficient length for successful homologous recombination with the endogenous gene in the genome of the targeted cell. Typically, several kilobases of flanking DNA (both at the 5′ and 3′ ends) are included in the vector. Methods for constructing homologous recombination vectors and homologous recombinant animals from recombinant stem cells are commonly known in the art (see, e.g., Thomas and Capecchi, 1987, Cell 51: 503; Bradley, 1991, Curr. Opin. Bio/Technol. 2: 823-29; and PCT Publication Nos. WO 90/11354, WO 91/01140, and WO 93/04169.

In a specific embodiment, the methods of Bonadio et al. (U.S. Pat. No. 5,942,496, entitled Methods and compositions for multiple gene transfer into bone cells, issued Aug. 24, 1999; and PCT WO95/22611, entitled Methods and compositions for stimulating bone cells, published Aug. 24, 1995) are used to introduce nucleic acids into a cell of interest, such as a stem cell, progenitor cell or exogenous cell cultured in the placenta, e.g., bone progenitor cells.

The cord blood-derived stem cells may be used, in specific embodiments, in autologous or heterologous enzyme replacement therapy to treat specific diseases or conditions, including, but not limited to lysosomal storage diseases, such as Tay-Sachs, Niemann-Pick, Fabry's, Gaucher's, Hunter's, and Hurler's syndromes, as well as other gangliosidoses, mucopolysaccharidoses, and glycogenoses.

In other embodiments, the cells may be used as autologous or heterologous transgene carriers in gene therapy to correct inborn errors of metabolism, adrenoleukodystrophy, cystic fibrosis, glycogen storage disease, hypothyroidism, sickle cell anemia, Pearson syndrome, Pompe's disease, phenylketonuria (PKU), porphyrias, maple syrup urine disease, homocystinuria, mucoplysaccharidenosis, chronic granulomatous disease and tyrosinemia and Tay-Sachs disease or to treat cancer, tumors or other pathological conditions.

In other embodiments, the cells may be used in autologous or heterologous tissue regeneration or replacement therapies or protocols, including, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, or for reconstruction of other damaged or diseased organs or tissues.

The large numbers of cord blood-derived stem cells and/or progenitor used in the methods of the invention would, in certain embodiments, reduce the need for large bone marrow donations. Approximately 1×10⁸ to 2×10⁸ bone marrow mononuclear cells per kilogram of patient weight must be infused for engraftment in a bone marrow transplantation (i.e., about 70 ml of marrow for a 70 kg donor). To obtain 70 ml requires an intensive donation and significant loss of blood in the donation process. In a specific embodiment, cells from a small bone marrow donation (e.g., 7-10 ml) could be expanded by propagation in a placental bioreactor before infusion into a recipient.

Furthermore, a small number of stem cells and progenitor cells normally circulate in the blood stream. In another embodiment, such exogenous stem cells or exogenous progenitor cells are collected by apheresis, a procedure in which blood is withdrawn, one or more components are selectively removed, and the remainder of the blood is reinfused into the donor.

In another embodiment, the administration of high doses of cord blood or cord blood derived stem cells is used as a supplemental treatment in addition to chemotherapy. Most chemotherapy agents used to target and destroy cancer cells act by killing all proliferating cells, i.e., cells going through cell division. Since bone marrow is one of the most actively proliferating tissues in the body, hematopoietic stem cells are frequently damaged or destroyed by chemotherapy agents and in consequence, blood cell production is diminishes or ceases. Chemotherapy must be terminated at intervals to allow the patient's hematopoietic system to replenish the blood cell supply before resuming chemotherapy. It may take a month or more for the formerly quiescent stem cells to proliferate and increase the white blood cell count to acceptable levels so that chemotherapy may resume (when again, the bone marrow stem cells are destroyed).

While the blood cells regenerate between chemotherapy treatments, however, the cancer has time to grow and possibly become more resistant to the chemotherapy drugs due to natural selection. Therefore, the longer chemotherapy is given and the shorter the duration between treatments, the greater the odds of successfully killing the cancer. To shorten the time between chemotherapy treatments, cord blood or cord blood-derived stem cells could be introduced into the patient. Such treatment would reduce the time the patient would exhibit a low blood cell count, and would therefore permit earlier resumption of the chemotherapy treatment.

### 4.3. Uses of Cord Blood and Cord Blood-Derived Stem Cells

Cord blood and cord blood-derived stem cells can be used for a wide variety of therapeutic protocols in which a tissue or organ of the body is augmented, repaired or replaced by the engraftment, transplantation or infusion of a desired cell population, such as a stem cell or progenitor cell population.

In a preferred embodiment of the invention, cord blood or cord blood-derived stem cells may be used as autologous and allogenic, including matched and mismatched HLA type hematopoietic transplants. In accordance with the use of cord blood or cord blood-derived stem cells as allogenic hematopoietic transplants, however, one may treat the host to reduce immunological rejection of the donor cells, such as those described in U.S. Pat. No. 5,800,539, issued Sep. 1, 1998; and U.S. Pat. No. 5,806,529, issued Sep. 15, 1998, both of which are incorporated herein by reference.

The cord blood or cord blood-derived stem cells can be used to repair damage of tissues and organs resulting from disease. In such an embodiment, a patient can be administered cord blood or cord blood-derived stem cells to regenerate or restore tissues or organs which have been damaged as a consequence of disease, e.g., enhance immune system following chemotherapy or radiation, repair heart tissue following myocardial infarction.

The cord blood or cord blood-derived stem cells can be used to augment or replace bone marrow cells in bone marrow transplantation. Human autologous and allogenic bone marrow transplantation are currently used as therapies for diseases such as leukemia, lymphoma and other life-threatening disorders. The drawback of these procedures, however, is that a large amount of donor bone marrow must be removed to insure that there is enough cells for engraftment.

The cord blood or cord blood-derived stem cells can provide stem cells and progenitor cells that would reduce the need for large bone marrow donation. It would also be, according to the methods of the invention, to obtain a small marrow donation and then expand the number of stem cells and progenitor cells culturing and expanding in the placenta before infusion or transplantation into a recipient.

The cord blood or cord blood-derived stem cells may be used, in specific embodiments, in autologous or heterologous enzyme replacement therapy to treat specific diseases or conditions, including, but not limited to lysosomal storage diseases, such as Tay-Sachs, Niemann-Pick, Fabry's, Gaucher's, Hunter's, Hurler's syndromes, as well as other gangliosidoses, mucopolysaccharidoses, and glycogenoses.

In other embodiments, the cells may be used as autologous or heterologous transgene carriers in gene therapy to correct inborn errors of metabolism such as adrenoleukodystrophy, cystic fibrosis, glycogen storage disease, hypothyroidism, sickle cell anemia, Pearson syndrome, Pompe's disease, phenylketonuria (PKU), and Tay-Sachs disease, porphyrias, maple syrup urine disease, homocystinuria, mucoplysaccharidenosis, chronic granulomatous disease, and tyrosinemia. or to treat cancer, tumors or other pathological or neoplastic conditions.

In other embodiments, the cells may be used in autologous or heterologous tissue regeneration or replacement therapies or protocols, including, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, scalp (hair) transplantation, or for reconstruction of other damaged or diseased organs or tissues.

Large amounts of cord blood, or large numbers of cord blood or cord blood-derived stem cells would, in certain embodiments, reduce the need for large bone marrow donations. Approximately 1×10⁸ to 2×10⁸ bone marrow mononuclear cells per kilogram of patient weight must be infused for engraftment in a bone marrow transplantation (i.e., about 70 ml of marrow for a 70 kg donor). To obtain 70 ml requires an intensive donation and significant loss of blood in the donation process. In a specific embodiment, cells from a small bone marrow donation (e.g., 7-10 ml) could be expanded by propagation in a placental bioreactor before infusion into a recipient.

In another embodiment, the cord blood or cord blood-derived stem cells can be used in a supplemental treatment in addition to chemotherapy. Most chemotherapy agents used to target and destroy cancer cells act by killing all proliferating cells, i.e., cells going through cell division. Since bone marrow is one of the most actively proliferating tissues in the body, hematopoietic stem cells are frequently damaged or destroyed by chemotherapy agents and in consequence, blood cell production is diminishes or ceases. Chemotherapy must be terminated at intervals to allow the patient's hematopoietic system to replenish the blood cell supply before resuming chemotherapy. It may take a month or more for the formerly quiescent stem cells to proliferate and increase the white blood cell count to acceptable levels so that chemotherapy may resume (when again, the bone marrow stem cells are destroyed).

While the blood cells regenerate between chemotherapy treatments, however, the cancer has time to grow and possibly become more resistant to the chemotherapy drugs due to natural selection. Therefore, the longer chemotherapy is given and the shorter the duration between treatments, the greater the odds of successfully killing the cancer. To shorten the time between chemotherapy treatments, cord blood or cord blood-derived stem cells could be introduced into the patient. Such treatment would reduce the time the patient would exhibit a low blood cell count, and would therefore permit earlier resumption of the chemotherapy treatment.

In another embodiment, the human placental stem cells can be used to treat or prevent genetic diseases such as chronic granulomatous disease.

### 4.4. Pharmaceutical Compositions

The present invention encompasses pharmaceutical compositions comprising a dose and/or doses effective upon single or multiple administration, prior to or following transplantation of conditioned or unconditioned human progenitor stem cells, exerting effect sufficient to inhibit, modulate and/or regulate the differentiation of human pluripotent and multipotent progenitor stem cells of placental origin into mesodermal and/or hematopoietic lineage cells.

In one embodiment, the invention provides pharmaceutical compositions that have high concentrations (or larger populations) of homogenous hematopoictic stem cells including but not limited to CD34+/CD38− cells; and CD34−/CD38− cells. One or more of these cell populations can be used with, or as a mixture with, other stem cells, for use in transplantation and other uses.

In a specific embodiment, cord blood or cord blood-derived stem cells are contained in a bag. In another embodiment, the invention provides cord blood or cord blood-derived stem cells that are “conditioned” before freezing.

In another embodiment, cord blood or cord blood-derived stem cells may be conditioned by the removal of red blood cells and/or granulocytes according to standard methods, so that a population of nucleated cells remains that is enriched for stem cells. Such an enriched population of stem cells may be used unfrozen, or frozen for later use. If the population of cells is to be frozen, a standard cryopreservative (e.g., DMSO, glycerol, Epilife™ Cell Freezing Medium (Cascade Biologics)) is added to the enriched population of cells before it is frozen.

In another embodiment, cord blood or cord blood-derived stem cells may be conditioned by the removal of red blood cells and/or granulocytes after it has been frozen and thawed.

According to the invention, agents that induce cell differentiation may be used to condition cord blood or cord blood-derived stem cells. In certain embodiments, an agent that induces differentiation can be added to a population of cells within a container, including, but not limited to, Ca²⁺, EGF, α-FGF, β-FGF, PDGF, keratinocyte growth factor (KGF), TGF-β, cytokines (e.g., IL-1α, IL-1β, IFN-γ, TFN), retinoic acid, transferrin, hormones (e.g., androgen, estrogen, insulin, prolactin, triiodothyronine, hydrocortisone, dexamethasone), sodium butyrate, TPA, DMSO, NMF, DMF, matrix elements (e.g., collagen, laminin, heparan sulfate, Matrigel™), or combinations thereof.

In another embodiment, agents that suppress cellular differentiation can be added to cord blood or cord blood-derived stem cells. In certain embodiments, an agent that suppresses differentiation can be added to a population of cells within a container, including, but not limited to, human Delta-1 and human Serrate-1 polypeptides (see, Sakano et al., U.S. Pat. No. 6,337,387 entitled “Differentiation-suppressive polypeptide”, issued Jan. 8, 2002), leukemia inhibitory factor (LIF), stem cell factor, or combinations thereof.

In certain embodiments, cord blood, or one or more populations of cord blood-derived stem cells are delivered to a patient in need thereof. In certain embodiments, two or more populations of fresh (never frozen) cells are delivered from a single container or single delivery system.

In another embodiment, two or more populations of frozen and thawed cells are delivered from a single container or single delivery system.

In another embodiment, each of two or more populations of fresh (never frozen) cells are transferred to, and delivered from, a single container or single delivery system. In another embodiment, each of two or more populations of frozen and thawed cells are transferred to, and delivered from, a single container or single delivery system. In another aspect of these embodiments, each population is delivered from a different IV infusion bag (e.g., from Baxter, Becton-Dickinson, Medcep, National Hospital Products or Terumo). The contents of each container (e.g., IV infusion bag) may be delivered via a separate delivery system, or each container may be “piggybacked” so that their contents are combined or mixed before delivery from a single delivery system.. For example, the two or more populations of cells may be fed into and/or mixed within a common flow line (e.g., tubing), or they may be fed into and/or mixed within a common container (e.g., chamber or bag).

According to the invention, the two or more populations of cells may be combined before administration, during or at administration or delivered simultaneously.

In one embodiment, a minimum of 1.7×10⁷ nucleated cells/kg is delivered to a patient in need thereof. Preferably, at least 2.5×10⁷ nucleated cells/kg is delivered to a patient in need thereof.

### 4.5. Methods of Treatment

In one embodiment, the invention provides a method of treating or preventing a disease or disorder in a subject comprising administering to a subject in which such treatment or prevention is desired a therapeutically effective amount of the stem cells of the invention.

In another embodiment, the invention provides a method of treating or preventing a disease or disorder in a subject comprising administering to a subject in which such treatment or prevention is desired a therapeutically effective amount of cord blood or cord blood-derived stem cells.

Cord blood or cord blood-derived stem cells are expected to have an anti-inflammatory effect when administered to an individual experiencing inflammation. In a preferred embodiment, cord blood or cord blood-derived stem cells may be used to treat any disease, condition or disorder resulting from, or associated with, inflammation. The inflammation may be present in any organ or tissue, for example, muscle; nervous system, including the brain, spinal cord and peripheral nervous system; vascular tissues, including cardiac tissue; pancreas; intestine or other organs of the digestive tract; lung; kidney; liver; reproductive organs; endothelial tissue, or endodermal tissue.

The cord blood or cord blood-derived stem cells may also be used to treat immune-related disorders, particularly autoimmune disorders, including those associated with inflammation. Thus, in certain embodiments, the invention provides a method of treating an individual having an autoimmune disease or condition, comprising administering to such individual a therapeutically effective amount of cord blood or cord blood-derived stem cells, wherein said disease or disorder can be, but is not limited to, diabetes, amylotrophic lateral sclerosis, myasthenia gravis, diabetic neuropathy or lupus. cord blood or cord blood-derived stem cells may also be used to treat acute or chronic allergies, e.g., seasonal allergies, food allergies, allergies to self-antigens, etc.

In certain embodiments, the disease or disorder includes, but is not limited to, any of the diseases or disorders disclosed herein, including, but not limited to aplastic anemia, myelodysplasia, myocardial infarction, seizure disorder, multiple sclerosis, stroke, hypotension, cardiac arrest, ischemia, inflammation, age-related loss of cognitive function, radiation damage, cerebral palsy, neurodegenerative disease, Alzheimer's disease, Parkinson's disease, Leigh disease, AIDS dementia, memory loss, amyotrophic lateral sclerosis (ALS), ischemic renal disease, brain or spinal cord trauma, heart-lung bypass, glaucoma, retinal ischemia, retinal trauma, lysosomal storage diseases, such as Tay-Sachs, Niemann-Pick, Fabry's, Gaucher's, Hunter's, and Hurler's syndromes, as well as other gangliosidoses, mucopolysaccharidoses, glycogenoses, inborn errors of metabolism, adrenoleukodystrophy, cystic fibrosis, glycogen storage disease, hypothyroidism, sickle cell anemia, Pearson syndrome, Pompe's disease, phenylketonuria (PKU), porphyrias, maple syrup urine disease, homocystinuria, mucoplysaccharidenosis, chronic granulomatous disease and tyrosinemia, Tay-Sachs disease, cancer, tumors or other pathological or neoplastic conditions.

In other embodiments, the cells may be used in the treatment of any kind of injury due to trauma, particularly trauma involving inflammation. Examples of such trauma-related conditions include central nervous system (CNS) injuries, including injuries to the brain, spinal cord, or tissue surrounding the CNS injuries to the peripheral nervous system (PNS); or injuries to any other part of the body. Such trauma may be caused by accident, or may be a normal or abnormal outcome of a medical procedure such as surgery or angioplasty. Trauma may also be the result of the rupture, failure or occlusion of a blood vessel, such as in a stroke or phlebitis. In specific embodiments, the cells may be used in autologous or heterologous tissue regeneration or replacement therapies or protocols, including, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, or for reconstruction of other damaged or diseased organs or tissues.

In a specific embodiment, the disease or disorder is aplastic anemia, myelodysplasia, leukemia, a bone marrow disorder or a hematopoietic disease or disorder. In another specific embodiment, the subject is a human.

In another embodiment, the invention provides a method of treating an individual having a disease, disorder or condition associated with or resulting from inflammation. In a specific embodiment, said disease, disorder or condition is a neurological disease, disorder or condition. In a more specific embodiment, said neurological disease is amylotrophic lateral sclerosis (ALS). In another more specific embodiment, said neurological disease is Parkinson's disease. In another specific embodiment, said disease is a vascular or cardiovascular disease. In a more specific embodiment, said disease is atherosclerosis. In another specific embodiment, said disease is diabetes.

A particularly useful aspect of cord blood or cord blood-derived stem cells is that there is no need to HLA-type the cells prior to administration. In other words, cord blood or cord blood-derived stem cells may be taken from a heterologous donor, or a plurality of heterologous donors, and transplanted to an individual in need of such cells, and the transplanted cells will remain within the host indefinitely. This elimination of the need for HLA typing greatly facilitates both the transplantation procedure itself and the identification of donors for transplantation. The cord blood or cord blood-derived stem cells may, however, be HLA-typed prior to administration.

The inventors have discovered that the efficacy of treating an individual with cord blood or cord blood-derived stem cells is enhanced if these cells are preconditioned. Preconditioning comprises storing the cells in a gas-permeable container of a period of time at approximately −5 to 23° C., 0 to 10° C., or, preferably, 4-5° C. The period of time may be between 18 hours and 21 days, between 48 hours and 10 days, and is preferably between 3-5 days. The cells may be cryopreserved prior to preconditioning or, preferably, are preconditioned immediately prior to administration.

Thus, in one embodiment, the invention provides a method of treating an individual comprising administering to said individual cord blood or cord blood-derived stem cells collected from at least one donor. “Donor” in this context means an adult, child, infant, or, preferably, a placenta. In another, preferred, embodiment, the method comprises administering to said individual cord blood or cord blood-derived stem cells that are collected from a plurality of donors and pooled. Alternatively, the cord blood or cord blood-derived stem cells may be taken from multiple donors separately, and administered separately, e.g., sequentially. In a specific embodiment, cord blood or cord blood-derived stem cells is taken from a plurality of donors and collected amounts (units) are administered on different days.

A particularly useful aspect of the invention is the administration of high doses of stem cells to an individual; such numbers of cells are significantly more effective than the material (for example, bone marrow or cord blood) from which they were derived. In this context, “high dose” indicates 5, 10, 15 or 20 times the number of total nucleated cells, including stem cells, particularly cord blood-derived stem cells, than would be administered, for example, in a bone marrow transplant. Typically, a patient receiving a stem cell infusion, for example for a bone marrow transplantation, receives one unit of cells, where a unit is approximately 1×10⁹ nucleated cells (corresponding to 1-2×10⁸ stem cells). For high-dose therapies, therefore, a patient would be administered at least 3 billion, 5 billion, 10 billion, 15 billion, 20 billion, 30 billion, 40 billion, 50 billion or more total nucleated cells, or, alternatively, at least 3 units, 5 units, 10 units, 20 units, 30 units, 40 units, 50 units or more. Thus, in one embodiment, the amount of cord blood or number of cord blood-derived stem cells administered to an individual corresponds to at least five times the number of nucleated cells normally administered in a bone marrow replacement. In another specific embodiment of the method, the amount of cord blood or number of cord blood-derived stem cells administered to an individual corresponds to at least ten times the number of nucleated cells normally administered in a bone marrow replacement. In another specific embodiment of the method, the amount of cord blood or number of cord blood-derived stem cells administered to an individual corresponds to at least fifteen times the number of nucleated cells normally administered in a bone marrow replacement. In another embodiment of the method, the total number of nucleated cells, which includes stem cells, administered to an individual is between 1-100×10⁸ per kilogram of body weight. In another embodiment, the number of total nucleated cells administered is at least 5 billion cells. In another embodiment, the total number of nucleated cells administered is at least 15 billion cells.

In another embodiment, said cord blood or cord blood-derived stem cells may be administered more than once. In another embodiment, said cord blood or cord blood-derived stem cells are preconditioned by storage from between 18 hours and 21 days prior to administration. In a more specific embodiment, the cells are preconditioned for 48 hours to 10 days prior to administration. In a preferred specific embodiment, said cells are preconditioned for 3-5 days prior to transplantation. In a preferred embodiment of any of the methods herein, said cord blood or cord blood-derived stem cells are not HLA typed prior to administration to an individual.

Treatment of an individual with cord blood or cord blood-derived stem cells may be considered efficacious if the disease, disorder or condition is measurably improved in any way. Such improvement may be shown by a number of indicators. Measurable indicators include, for example, detectable changes in a physiological condition or set of physiological conditions associated with a particular disease, disorder or condition (including, but not limited to, blood pressure, heart rate, respiratory rate, counts of various blood cell types, levels in the blood of certain proteins, carbohydrates, lipids or cytokines or modulation expression of genetic markers associated with the disease, disorder or condition). Treatment of an individual with the stem cells or supplemented cell populations of the invention would be considered effective if any one of such indicators responds to such treatment by changing to a value that is within, or closer to, the normal value. The normal value may be established by normal ranges that are known in the art for various indicators, or by comparison to such values in a control. In medical science, the efficacy of a treatment is also often characterized in terms of an individual's impressions and subjective feeling of the individual's state of health. Improvement therefore may also be characterized by subjective indicators, such as the individual's subjective feeling of improvement, increased well-being, increased state of health, improved level of energy, or the like, after administration of the stem cells or supplemented cell populations of the invention.

The cord blood or cord blood-derived stem cells may be administered to a patient in any pharmaceutically or medically acceptable manner, including by injection or transfusion. The cells or supplemented cell populations may be contain, or be contained in any pharmaceutically-acceptable carrier. The cord blood or cord blood-derived stem cells may be carried, stored, or transported in any pharmaceutically or medically acceptable container, for example, a blood bag, transfer bag, plastic tube or vial.

### 4.6. Kits

The invention also provides a pharmaceutical pack or kit comprising one or more containers filled with one or more of the ingredients of the pharmaceutical compositions of the invention. Optionally associated with such container(s) can be: an apparatus for cell culture, one or more containers filled with a cell culture medium or one or more components of a cell culture medium, an apparatus for use in delivery of the compositions of the invention, e.g., an apparatus for the intravenous injection of the compositions of the invention, and/or a notice in the form prescribed by a governmental agency regulating the manufacture, use or sale of pharmaceuticals or biological products, which notice reflects approval by the agency of manufacture, use or sale for human administration.

The following experimental examples are offered by way of illustration and not by way of limitation.

### 5. EXAMPLES

### 5.1 Example 1

### Induction of Differentiation into Particular Cell Types

Cord blood cells and/or are induced to differentiate into a particular cell type by exposure to a growth factor. Growth factors that are used to induce induction include, but are not limited to: GM-CSF, IL-4, Flt3L, CD40L, IFN-alpha, TNF-alpha, IFN-gamma, IL-2, IL-6, retinoic acid, basic fibroblast growth factor, TGF-beta-1, TGF-beta-3, hepatocyte growth factor, epidermal growth factor, cardiotropin-1, angiotensinogen, angiotensin I (AI), angiotensin II (AII), AII AT₂ type 2 receptor agonists, or analogs or fragments thereof.

### 5.1.1 Induction of Differentiation into Neurons

This example describes the induction of cord blood cells to differentiate into neurons. The following protocol is employed to induce neuronal differentiation:
1. Stem cells are grown for 24 hr in preinduction media consisting of DMEM/20% FBS and 1 mM beta-mercaptoethanol.
2. Preinduction media is removed and cells are washed with PBS.
3. Neuronal induction media consisting of DMEM and 1-10 mM betamercaptoethanol is added. Alternatively, induction media consisting of DMEM/2% DMSO/200 μM butylated hydroxyanisole may be used to enhance neuronal differentiation efficiency.
4. In certain embodiments, morphologic and molecular changes may occur as early as 60 minutes after exposure to serum-free media and betamercaptoethanol (Woodbury et al., J. Neurosci. Res., 61:364-370). RT/PCR may be used to assess the expression of e.g., nerve growth factor receptor and neurofilament heavy chain genes.

### 5.1.2 Induction of Differentiation into Adipocytes

This example describes the induction of cord blood cells to differentiate into adipocytes. The following protocol is employed to induce adipogenic differentiation:
1. Stem cells are grown in MSCGM (Bio Whittaker) or DMEM supplemented with 15% cord blood serum.
2. Three cycles of induction/maintenance are used. Each cycle consists of feeding the placental stem cells with Adipogenesis Induction Medium (Bio Whittaker) and culturing the cells for 3 days (at 37° C., 5% CO₂), followed by 1-3 days of culture in Adipogenesis Maintenance Medium (Bio Whittaker). An induction medium is used that contains 1 μM dexamethasone, 0.2 mM indomethacin, 0.01 mg/ml insulin, 0.5 mM IBMX, DMEM-high glucose, FBS, and antibiotics.
3. After 3 complete cycles of induction/maintenance, the cells are cultured for an additional 7 days in adipogenesis maintenance medium, replacing the medium every 2-3 days.
4. Adipogenesis may be assessed by the development of multiple intracytoplasmic lipid vesicles that can be easily observed using the lipophilic stain oil red O. RT/PCR assays are employed to examine the expression of lipase and fatty acid binding protein genes.

### 5.1.3 Induction of Differentiation into Chondrocytes

This example describes the induction of cord blood cells to differentiate into chondrocytes. The following protocol is employed to induce chondrogenic differentiation:
1. Stem cells are maintained in MSCGM (Bio Whittaker) or DMEM supplemented with 15% cord blood serum.
2. Stem cells are aliquoted into a sterile polypropylene tube. The cells are centrifuged (150×g for 5 minutes), and washed twice in Incomplete Chondrogenesis Medium (Bio Whittaker).
3. After the last wash, the cells are resuspended in Complete Chondrogenesis Medium (Bio Whittaker) containing 0.01 μg/ml TGF-beta-3 at a concentration of 5×10(5) cells/ml.
4. 0.5 ml of cells is aliquoted into a 15 ml polypropylene culture tube. The cells are pelleted at 150×g for 5 minutes. The pellet is left intact in the medium.
5. Loosely capped tubes are incubated at 37° C., 5% CO₂ for 24 hours.
6. The cell pellets are fed every 2-3 days with freshly prepared complete chondrogenesis medium.
7. Pellets are maintained suspended in medium by daily agitation using a low speed vortex.
8. Chondrogenic cell pellets are harvested after 14-28 days in culture.
9. Chondrogenesis may be characterized by e.g., observation of production of esoinophilic ground substance, assessing cell morphology, an/or RT/PCR for examining collagen 2 and collagen 9 gene expression.

### 5.1.4 Induction of Differentiation into Osteocytes

This example describes the induction of cord blood cells to differentiate into osteocytes. The following protocol is employed to induce osteogenic differentiation:
1. Adherent cultures of cord blood-derived stem cells are cultured in MSCGM (Bio Whittaker) or DMEM supplemented with 15% cord blood serum.
2. Cultures are rested for 24 hours in tissue culture flasks.
3. Osteogenic differentiation is induced by replacing MSCGM with Osteogenic Induction Medium (Bio Whittaker) containing 0.1 μM dexamethasone, 0.05 mM ascorbic acid-2-phosphate, 10 mM beta glycerophosphate.
4. Cells are fed every 3-4 days for 2-3 weeks with Osteogenic Induction Medium.
5. Differentiation is assayed using a calcium-specific stain and RT/PCR for alkaline phosphatase and osteopontin gene expression.

### 5.1.5 Induction of Differentiation into Hepatocytes

This example describes the induction of cord blood cells to differentiate into hepatocytes. The following protocol is employed to induce hepatogenic differentiation:
1. Cord blood-derived stem cells are cultured in DMEM/20% CBS supplemented with hepatocyte growth factor, 20 ng/ml; and epidermal growth factor, 100 ng/ml. KnockOut Serum Replacement may be used in lieu of FBS.
2. IL-6 50 ng/ml is added to induction flasks.

### 5.1.6 Induction of Differentiation into Pancreatic Cells

This example describes the induction of cord blood cells to differentiate into pancreatic cells. The following protocol is employed to induce pancreatic differentiation:
1. Cord blood-derived stem cells are cultured in DMEM/20% CBS, supplemented with basic fibroblast growth factor, 10 ng/ml; and transforming growth factor beta-1, 2 ng/ml. KnockOut Serum Replacement may be used in lieu of CBS.
2. Conditioned media from nestin-positive neuronal cell cultures is added to media at a 50/50 concentration.
3. Cells are cultured for 14-28 days, refeeding every 3-4 days.
4. Differentiation is characterized by assaying for insulin protein or insulin gene expression by RT/PCR.

### 5.1.7 Induction of Differentiation into Cardiac Cells

This example describes the induction of cord blood cells to differentiate into cardiac cells. The following protocol is employed to induce myogenic differentiation:
1. Cord blood-derived stem cells are cultured in DMEM/20% CBS, supplemented with retinoic acid, 1 μM; basic fibroblast growth factor, 10 ng/ml; and transforming growth factor beta-1, 2 ng/ml; and epidermal growth factor, 100 ng/ml. KnockOut Serum Replacement may be used in lieu of CBS.
2. Alternatively, stem cells are cultured in DMEM/20% CBS supplemented with 50 ng/ml Cardiotropin-1 for 24 hours.
3. Alternatively, stem cells are maintained in protein-free media for 5-7 days, then stimulated with human myocardium extract (escalating dose analysis). Myocardium extract is produced by homogenizing 1 gm human myocardium in 1% HEPES buffer supplemented with 1% cord blood serum. The suspension is incubated for 60 minutes, then centrifuged and the supernatant collected.
4. Cells are cultured for 10-14 days, refeeding every 3-4 days.
5. Differentiation is assessed using cardiac actin RT/PCR gene expression assays.

### 5.1.8 Characterization of Cord Blood Cells Prior to and/or After Differentiation

The cord blood cells are characterized prior to and/or after differentiation by measuring changes in morphology and cell surface markers using techniques such as flow cytometry and immunocytochemistry, and measuring changes in gene expression using techniques, such as PCR. Cells that have been exposed to growth factors and/or that have differentiated are characterized by the presence or absence of the following cell surface markers: CD10+, CD29+, CD34−, CD38−, CD44+, CD45−, CD54+, CD90+, SH2+, SH3+, SH4+, SSEA3−, SSEA4−, OCT-4+, and ABC-p+. Preferably, the cord blood-derived stem cell are characterized, prior to differentiation, by the presence of cell surface markers OCT-4+, APC-p+, CD34− and CD38−. Stem cells bearing these markers are as versatile (e.g., pluripotent) as human embryonic stem cells. Cord blood cells are characterized, prior to differentiation, by the presence of cell surface markers CD34+ and CD38+. Differentiated cells derived from cord blood cells preferably do not express these markers.

### 5.2 Example 2

### Treatment of Individuals Having Amylotrophic Lateral Sclerosis with Cord Blood or Cord Blood-Derived Stem Cells

Amyotrophic Lateral Sclerosis (ALS), also called Lou Gehrig's disease, is a fatal neurodegenerative disease affecting motor neurons of the cortex, brain stem and spinal cord. ALS affects as many as 20,000 Americans with 5,000 new cases occurring in the US each year. The majority of ALS cases are sporadic (S-ALS) while ˜5-10% are hereditary (familial—F-ALS). ALS occurs when specific nerve cells in the brain and spinal cord that control voluntary movement gradually degenerate. The cardinal feature of ALS is the loss of spinal motor neurons which causes the muscles under their control to weaken and waste away leading to paralysis. ALS manifests itself in different ways, depending on which muscles weaken first. ALS strikes in mid-life with men being one-and-a-half times more likely to have the disease as women. ALS is usually fatal within five years after diagnosis.

ALS has both familial and sporadic forms, and the familial forms have now been linked to several distinct genetic loci. Only about 5-10% of ALS cases are familial. Of these, 15-20% are due to mutations in the gene encoding Cu/Zn superoxide dismutase 1 (SOD1). These appear to be “gain-of-function” mutations that confer toxic properties on the enzyme. The discovery of SOD mutations as a cause for ALS has paved the way for some progress in the understanding of the disease; animal models for the disease are now available and hypotheses are being developed and tested concerning the molecular events leading to cell death.

Presented below is an example method of treating an individual having ALS with cord blood or cord blood-derived stem cells. The method involves intravenous infusion through a peripheral, temporary angiocatheter.

An individual having ALS is first assessed by the performance of standard laboratory analyses. Such analyses may include a metabolic profile; CDC with differential; lipid profile; fibrinogen level; ABO rH typing of the blood; liver function tests; and determination of BUN/creatine levels. Individuals are instructed the day prior to the transplant to take the following medications: diphenhydramine (Benadryl™), 25 mg t.i.d, and prednisone, 10 mg.

Cord blood is taken, or cord blood-derived stem cells are taken, from cryopreserved stock, thawed, and maintained for approximately two days prior to transplantation at a temperature of approximately 5° C.

The individual is transplanted at an outpatient clinical center which has all facilities necessary for intravenous infusion, physiological monitoring and physical observation. Approximately one hour prior to transplantation, the individual receives diphenhydramine (Benadryl™), 25 mg×1 P.O., and prednisone, 10 mg×1 P.O. This is precautionary, and is meant to reduce the likelihood of an acute allergic reaction. At the time of transfusion, an 18 G indwelling peripheral venous line is places into one of the individual's extremities, and is maintained open by infusion of D5 ½ normal saline+20 mEq KCl at a TKO rate. The individual is examined prior to transplantation, specifically to note heart rate, respiratory rate, temperature. Other monitoring may be performed, such as an electrocardiogram and blood pressure measurement.

Cord blood or cord blood-derived stem cells are then infused at a rate of 1 unit per hour in a total delivered fluid volume of 60 ml, where a unit is approximately 1-2×10⁹ total nucleated cells. Alternatively, the unit of cord blood or cord blood-derived stem cells is delivered in a total fluid volume of 60 ml. Based upon data from pre-clinical studies in mice, a total of 2.0-2.5×10⁸ cells per kilogram of body weight should be administered. For example, a 70 kilogram individual would receive approximately 14-18×10⁹ total nucleated cells. The individual should be monitored for signs of allergic response or hypersensitivity, which are signals for immediate cessation of infusion.

Post-infusion, the individual should be monitored in a recumbent position for at least 60 minutes, whereupon he or she may resume normal activities.

### 5.3 Example 3

### Treatment of Individuals Having Atherosclerosis Using Cord Blood or Cord Blood-Derived Stem Cells

The infusion protocol outlined in Example 2 may be used to administer the cord blood or cord blood-derived stem cells to a patient having atherosclerosis. Cord blood or cord blood-derived stem cells may be administered to asymptomatic individuals, individuals that are candidates for angioplasty, or to patients that have recently (within one week) undergone cardiac surgery.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

1. A method of treating an individual comprising administration of a composition comprising cord blood or cord blood-derived stem cells, wherein said administration delivers at least 1×10¹⁰ total nucleated cells, or at least 1×10⁹ stem cells, to an individual in need of said administration.

2. The method of claim 1 wherein the cord blood or cord blood-derived stem cells are suitable for bone marrow transplantation.

3. The method of claim 2 wherein the cord blood or cord blood-derived stem cells are suitable for administration in humans.

4. The method of claim 2 wherein a plurality of the cord blood-derived stem cells express the cell surface markers CD34⁺ and CD38⁻.

5. The method of claim 2 wherein a plurality of the umbilical cord blood stem cells express the cell surface markers CD34+ and CD38+.

6. The method of claim 2 additionally comprising contacting the cord blood or cord blood-derived stem cells with a growth factor.

7. The method of claim 6 wherein the growth factor is a cytokine, lymphokine, interferon, colony stimulating factor (CSF), interferon, chemokine, interleukin, human hematopoietic growth factor, hematopoietic growth factor ligand, stem cell factor, thrombopoietin (Tpo), granulocyte colony-stimulating factor (G-CSF), leukemia inhibitory factor, basic fibroblast growth factor, placenta derived growth factor or epidermal growth factor.

8. The method of claim 6 wherein the cord blood or cord blood-derived stem cells are contacted with the growth factor to induce differentiation into a plurality of cell types.

9. The method of claim 6 wherein the cord blood or cord blood-derived stem cells are contacted with the growth factor to prevent or suppress differentiation into a particular cell type.

10. A method of treating myelodysplasia which comprises administering cord blood or cord blood-derived stem cells to a patient in need thereof.

11. The method of claim 1 wherein said administration delivers at least 3×10¹⁰ total nucleated cells or at least 3×10⁹ stem cells.

12. (canceled)

13. The method of claim 1 wherein said administration delivers at least 2×10¹⁰ total nucleated cells or at least 2×10⁹ stem cells.

14. The method of claim 1 wherein said individual has a disease, disorder or condition that includes an inflammation component.

15. The method of claim 1 wherein said individual has a vascular disease, disorder or condition.

16. The method of claim 15 wherein said disease, disorder or condition is atherosclerosis.

17. The method of claim 1 wherein said individual has a neurological disease, disorder or condition.

18. The method of claim 17, wherein said disease, disorder or condition is selected from the group consisting of amyotrophic lateral sclerosis and multiple sclerosis.

19. (canceled)

20. (canceled)

21. The method of claim 1, wherein said individual has undergone a trauma or injury.

22. The method of claim 21, where said trauma or injury is trauma or injury to the central nervous system.

23. The method of claim 21, wherein said trauma or injury is trauma or injury to the peripheral nervous system.

24. The method of claim 1, wherein said at least 1×10¹⁰ total nucleated cells, or at least 1×10⁹ stem cells, comprises cells derived from a plurality of donors.

25. The method of claim 1 wherein none of said cells in said composition is HLA-typed prior to said administration.

26. The method of claim 1 wherein said composition is preconditioned for between 18 hours and 21 days prior to said administration.

27. The method of claim 1 wherein said composition is preconditioned for between 48 hours and 10 days prior to said administration.

28. The method of claim 1, wherein said composition is preconditioned for between 3-5 days prior to said administration.
